(19) United States
(12) Patent Application Publication
(10) Pub. No.: US 2016/0242727 A1
(43) Pub. Date: Aug. 25, 2016

(54) AUTOMATED ADJUSTMENT OF SIGNAL ANALYSIS PARAMETERS FOR X-RAY DETECTORS

(71) Applicant: Siemens Aktiengesellschaft, München (DE)

(72) Inventors: Thomas FLOHR, Uehlfeld (DE); Steffen KAPPLER, Effeltrich (DE)

(21) Appl. No.: 15/012,892

(22) Filed: Feb. 2, 2016

(30) Foreign Application Priority Data
Feb. 19, 2015 (DE) 102015202999.9

(51) Int. Cl. A61B 6/00

(52) U.S. Cl. CPC A61B 6/545; A61B 6/544

(57) ABSTRACT

A method for the automated determination of an adjusted setting for signal analysis parameters of an x-ray detector is described. With an embodiment of the method, information relating to the dimensions of the object to be examined, the x-ray attenuation in the object to be examined, the nature of the examination and the examination region of the object to be examined is acquired. Signal analysis parameter values are then determined based on the acquired information. A method for automatically setting signal analysis parameters of an x-ray detector is also described. A facility for determining an adjusted setting for signal analysis parameters of an x-ray detector is also described. An x-ray system is also described.

### PRIORITY STATEMENT

The present application hereby claims priority under 35 U.S.C. §119 to German patent application number DE 102015202999.9 filed Feb. 19, 2015, the entire contents of which are hereby incorporated herein by reference.

### FIELD

The invention generally relates to a method for automatically determining an adjusted setting for signal analysis parameters of an x-ray detector. The invention also generally relates to a method for automatically setting signal analysis parameters of an x-ray detector. The invention further generally relates to a facility for determining an adjusted setting for signal analysis parameters of an x-ray detector. The invention also generally relates to an x-ray detector.

### BACKGROUND

Two-dimensional or three-dimensional image data is frequently generated with the aid of state-of-the-art imaging methods, such data allowing the visualization of an imaged examination object and in addition also being able to be used for further applications.

The imaging methods are frequently based on the detection of x-ray radiation, with what is referred to as projection measurement data being generated in the process. Projection measurement data can be acquired with the aid of a computed tomography system (CT system) for example. In CT systems, a combination consisting of x-ray source and oppositely positioned x-ray detector is arranged on a gantry and typically rotates around a measurement space in which the examination object (referred to hereinafter without loss of generality as the patient) is situated. In this case the center of rotation (also known as the isocenter) coincides with an axis referred to as system axis z. In the course of one or more rotations the patient is irradiated with x-ray radiation from the x-ray source, projection measurement data or x-ray projection data being acquired with the aid of the x-ray detector opposite.

The generated projection measurement data is dependent in particular on the design of the x-ray detector. X-ray detectors typically have a plurality of detection units which in most cases are arranged in the form of a regular pixel array. Each of the detection units generates a detection signal for x-ray radiation striking the detection units, the detection signal being analyzed in respect of intensity and spectral distribution of the x-ray radiation at specific time points in order to obtain inferences in relation to the examination object and to generate projection measurement data.

In the case of what are termed quanta-counting or photon-counting x-ray detectors, the detection signal for x-ray radiation is analyzed in respect of the intensity and the spectral distribution of the x-ray radiation in the form of count rates. The count rates are made available as output data of what is referred to as a detector channel which is assigned to one detection unit in each instance. With quanta-counting or photon-counting detectors having a number of energy thresholds, each detector channel normally generates a set of count rates per projection based on the respective detection signal of the detection unit. In such cases the set of count rates can include count rates for a number of different, in particular simultaneously checked energy threshold values. The energy threshold values and the number of energy thresholds to which an energy threshold value is assigned in each instance are in most cases predefined as signal analysis parameters for the acquisition of the projection.

The quality of the generated projection measurement data here is influenced inter alia by the separation of two x-ray radiation quanta in the detection signal which is possible in a temporal interval referred to as “single pulse separation”. Furthermore, the quality of the projection measurement data can also be influenced by the energetic interval in which the separation of two x-ray radiation quanta (which is generally represented as voltage distance in the signal) is possible.

The position of the energy thresholds can be adjusted in the quanta-counting x-ray detectors by changing this settable signal analysis parameter and can be changed from recording to recording if required. With a typical quanta-counting detector for example energy thresholds with the values 25 keV, 35 keV, 55 keV and 80 keV are used.

A further type of signal analysis parameters is what are known as “signal shape parameters”, also referred to as “shaping parameters”, for example a “shaping time”, what is referred to as an “undershoot” or what is referred to as a “gain”. These physical variables are shown in a graph in FIG. 1.

“Shaping time” (also: “peaking time”) is the time during which the charge carriers generated in the detector can contribute to the pulse shape of an individual detection pulse. As mentioned above, the detection signal is typically a charge or current pulse, which is converted to a voltage pulse with the aid of the analysis module. The “shaping time” therefore relates to a time period in which charge is accumulated on the detection surface to generate an individual voltage pulse and the voltage pulse is shaped. The “shaping time” is typically in the range between 5 ns and 1 μs.

What is known as “undershoot” is a voltage value (generally preceded by a sign opposite to that of the signal pulse), to which the generated voltage signal drops, before it returns to its quiescent level. Undershoot can be used in particular to improve the signal separation of different voltage pulses that follow quickly one after the other.

What is known as the amplification factor or “gain” determines the ratio between the accumulated charge or input current strength and the size of the corresponding voltage pulse. It therefore determines the maximum output signal level, in other words for example at the output of the amplifier, which is reached when a current pulse is amplified.

The described signal shape parameters allow a choice to be made in particular between a preference for a precise charge measurement and a preference for a precise separation of x-ray quanta. If a long shaping time is chosen for example, precise charge measurement and therefore precise energy determination are possible. In other words the preference here is for a precise determination of the x-ray spectrum.

If however a large number of x-ray quanta have to be analyzed at approximately the same time, in other words what is known as an instance of “high flux”, it is not always possible to separate the resulting pulses for x-ray quanta that follow one another immediately with a long shaping time. Reference is made to what are known as “pile-up events”, in which the generated voltage pulses of x-ray quanta absorbed immediately after one another can no longer be separated. In other words it is possible in particular to set the number of “pile-up events” by way of the shaping time mentioned above. If the number of “pile-up events” is reduced, for example by way of a short “shaping time” for the high flux instance, it may however reduce the precision of the charge measurement with the generated voltage pulse. This is referred to as a “ballistic deficit”. This phenomenon reduces the precision of a measurement of the energy spectrum. The choice of a defined “shaping time” is therefore always a compromise between achievable charge separation, in other words measurement of the x-ray intensity, and precision of the charge determination, in other words measurement of the spectral distribution of the x-ray radiation.

The values of the optimum signal shape parameters can be a function for example of the size of a patient to be examined. If the patient is very large for example, the incident x-ray radiation will be attenuated to a greater degree and a much reduced intensity will strike the x-ray detector. This is an instance of low flux. In this situation a longer shaping time should be set to allow sufficiently precise charge measurement and therefore precise energy determination.

If however a small patient is examined, the incident x-ray radiation will be attenuated to a much lesser degree and a higher intensity strikes the x-ray detector. This is an instance of high flux. In this situation a shorter shaping time should be set in order to be able to separate the individual signal events from one another and thus to be able to determine the x-ray intensity of the incident radiation sufficiently precisely.

### SUMMARY

The inventors have recognized that it is important therefore also to adjust the shape of the electrical signals triggered by the counter events for a specific recording with the aid of the signal shape parameters. Energy threshold values and signal shape parameters will be referred to together in the following as signal analysis parameters.

Depending on the type of CT examination being performed, the materials contained in the object to be examined and the size and shape of the examination object, it is advantageous for the subsequent quality of the data and its subsequent evaluation to set energy thresholds to different parameters values and to adjust the signal shape parameters accordingly.

The signal analysis parameters are conventionally set in a manual manner. Either the signal analysis parameters are set permanently once or they are determined and set manually by an experienced operator before a corresponding measurement for example as a function of a specific protocol, an estimated patient size and the nature of the examination of the patient. This procedure requires considerable experience, knowledge and skill and also requires a certain time, thereby extending the overall time required to examine the patient.

An embodiment of the present invention allows a simplified, shortened and more precise adjustment of x-ray detectors.

An embodiment is directed to a method for automatically determining an adjusted setting for signal analysis parameters of an x-ray detector; a method for automatically setting signal analysis parameters of an x-ray detector; a facility for determining an adjusted setting for signal analysis parameters of an x-ray detector; and an x-ray system.

With an embodiment of the inventive method for the automated determination of an adjusted setting for signal analysis parameters of an x-ray detector information relating to at least one of the following groups of examination parameters is acquired:
the dimensions of an object to be examined,
the x-ray attenuation in the object to be examined,
the nature of the examination of the object to be examined, and
the examination region of the object to be examined.

Signal analysis parameter values are then determined in an automated manner based on the acquired information.

With an embodiment of the inventive method for automatically setting signal analysis parameters of an x-ray detector, an embodiment of the inventive method for the automated determination of an adjusted setting for signal analysis parameters of an x-ray detector is first performed. The signal analysis parameters of the x-ray detector are then set in an automated manner based on the determined signal analysis parameter values.

An embodiment of the inventive facility is disclosed, for determining an adjusted setting for signal analysis parameters of an x-ray detector. The facility includes an input interface for acquiring information relating to at least one of the following groups of examination parameters:
the dimensions of an object to be examined,
the x-ray attenuation in the object to be examined,
the nature of the examination of the object to be examined, and
the examination region of the object to be examined.

An embodiment of the inventive facility also comprises a determination unit for the automated determination of signal analysis parameter values based on the acquired information.

An embodiment of the inventive x-ray system features an embodiment of the inventive facility for determining an adjusted setting for signal analysis parameters of an x-ray detector.

A largely software-based implementation has the advantage that control facilities already used to date can also be upgraded in a simple manner by way of a software update, in order to operate in the inventive manner. To this extent the object is also achieved by a non-transitory computer readable medium, which can be loaded directly into a storage facility of an x-ray system, having program segments for executing all the steps of an embodiment of the inventive method when the program is executed in the storage facility.

The claims and the description that follows in each instance contain particularly advantageous embodiments and developments of the invention. In particular the claims of one claim category can also be developed in the same manner as the dependent claims of another claim category. Also the different features of different example embodiments and claims can be combined to result in new example embodiments within the scope of the invention.

### BRIEF DESCRIPTION OF THE DRAWINGS

The invention is described again in more detail below based on example embodiments with reference to the accompanying figures, in which:

FIG. 1 shows the temporal profile of a generated voltage signal, which is generated using a number of signal analysis parameters,

FIG. 2 shows a schematic diagram of a CT device with an adjustment facility according to one example embodiment of the invention,

FIG. 3 shows a block circuit diagram of an adjustment facility according to one example embodiment of the invention,

FIG. 4 shows a flow diagram representing one example embodiment of the inventive method for the automated determination of an adjusted setting for signal analysis parameters of an x-ray detector,

FIG. 5 shows a flow diagram representing one example embodiment of the inventive method for automatically setting signal analysis parameters of an x-ray detector.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Further, although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

With an embodiment of the inventive method for the automated determination of an adjusted setting for signal analysis parameters of an x-ray detector information relating to at least one of the following groups of examination parameters is acquired:
the dimensions of an object to be examined,
the x-ray attenuation in the object to be examined,
the nature of the examination of the object to be examined, and
the examination region of the object to be examined.

Signal analysis parameter values are then determined in an automated manner based on the acquired information.

X-ray attenuation here refers to the attenuation of the x-ray radiation during an x-ray recording due to absorption by the object to be examined.

In the following a parameter refers to a settable variable, for example variables characterizing energy thresholds or signal shape. A parameter value in contrast refers to a specific value for the variables.

At least the dimensions of the object to be examined or the x-ray attenuation in the object to be examined are preferably acquired as examination parameters.

At least the dimensions of the object to be examined and the x-ray attenuation in the object to be examined are particularly preferably acquired as the examination parameters.

Automated adjustment accelerates the setting of signal analysis parameters of an x-ray detector and makes it more precise. The challenge for operators, who do not have to intervene in the adjustment process and also no longer require specialist technical knowledge in this field to perform the setting of an x-ray system, is also reduced.

With an embodiment of the inventive method for automatically setting signal analysis parameters of an x-ray detector, an embodiment of the inventive method for the automated determination of an adjusted setting for signal analysis parameters of an x-ray detector is first performed. The signal analysis parameters of the x-ray detector are then set in an automated manner based on the determined signal analysis parameter values.

An embodiment of the inventive facility is disclosed, for determining an adjusted setting for signal analysis parameters of an x-ray detector. The facility includes an input interface for acquiring information relating to at least one of the following groups of examination parameters:
the dimensions of an object to be examined,
the x-ray attenuation in the object to be examined,
the nature of the examination of the object to be examined, and
the examination region of the object to be examined.

An embodiment of the inventive facility also comprises a determination unit for the automated determination of signal analysis parameter values based on the acquired information.

The input interface is preferably set up to acquire at least the dimensions of the object to be examined or the x-ray attenuation in the object to be examined as examination parameters.

The input interface is particularly preferably set up to acquire at least the dimensions of the object to be examined and the x-ray attenuation in the object to be examined as examination parameters.

An embodiment of the inventive x-ray system features an embodiment of the inventive facility for determining an adjusted setting for signal analysis parameters of an x-ray detector.

The signal analysis parameter values can be determined for example with the aid of an optimization method. In this process a model function, the variables of which represent the signal analysis parameters, is optimized taking account of the acquired information as parameters of the model function.

The term “x-ray system” refers in particular to a computed tomography system but can also include a simple x-ray device or an angiography device.

In the case of an x-ray computed tomography system, an embodiment of an inventive x-ray-computed tomography system has a projection data acquisition unit. The projection data acquisition unit comprises an x-ray source and a detector system for acquiring projection measurement data of an object and additionally the inventive facility for determining an adjusted setting for signal analysis parameters of an x-ray detector.

The key components of an embodiment of the inventive facility for determining an adjusted setting for signal analysis parameters of an x-ray detector can be configured for the greatest part in the form of software components. This is true in particular of the determination unit. In principle some of these components can however also be implemented in the form of software-assisted hardware, for example FPGAs or the like, in particular when particularly fast calculations are required. Similarly the required interfaces can be configured as software interfaces for example when data only has to be taken from other software components. They can however also be configured as interfaces configured in the manner of hardware which are controlled by suitable software.

The facility for determining an adjusted setting for signal analysis parameters of an x-ray detector can in particular be part of a user terminal or a control facility of a CT system.

A largely software-based implementation has the advantage that control facilities already used to date can also be upgraded in a simple manner by way of a software update, in order to operate in the inventive manner. To this extent the object is also achieved by a computer program product or non-transitory computer readable medium, which can be loaded directly into a storage facility of an x-ray system, having program segments for executing all the steps of an embodiment of the inventive method when the program is executed in the storage facility.

In one embodiment of the inventive method for the automated determination of an adjusted setting for signal analysis parameters of an x-ray detector the dimensions of the object to be examined comprise its size and/or shape. The size of an object to be examined is for example significant for the attenuation of the x-ray beam and therefore influences the dose of radiation incident on the detector. As mentioned above, different values are selected for the signal shape parameters depending on whether there is “high flux” or “low flux”. The shape of the object to be examined, associated for example with the thickness of the object to be examined, influences the attenuation of the x-ray beams occurring during the recording of the projection.

However a dependency of the attenuation on the energy spectrum of the radiation additionally occurs with the attenuation of the x-ray dose. Lower-energy radiation is attenuated to a greater degree than higher-energy radiation. For this reason the x-ray spectrum is hardened, in particular with thick objects to be examined, in other words the harder, higher-energy x-ray radiation is attenuated less by the object to be examined. Such hardening of the x-ray spectrum can be taken into account for example when selecting the energy thresholds. As higher-energy radiation is detected first and foremost, it is expedient to select the energy thresholds with finer resolution in the region of higher-energy radiation.

In one preferred embodiment of the inventive method for the automated determination of an adjusted setting for signal analysis parameters of an x-ray detector, the signal analysis parameters comprise parameters for energy thresholds and/or signal shape parameters.

The acquisition of the information particularly preferably comprises the recording of a topogram of the object to be examined. Information relating to the dimensions of the object to be examined and the x-ray attenuation produced by the object to be examined can be obtained based on the topogram recording.

Additionally or alternatively the acquisition of the information can also comprise a photographic recording of the object to be examined. In an alternative acquisition of the information with the aid of a photographic recording the dose exposure of the object to be examined, for example a patient, can be reduced.

Additionally the acquisition of information can comprise the weighing of the object to be examined. Weighing can be performed for example with the aid of an automatic scale built into a patient couch. The weight can be correlated for example with the thickness and width of the patient and thus indirectly provide information about x-ray attenuation by the patient and a signal shape parameter to be selected.

The acquisition of information can comprise for example the receiving of data relating to the object to be examined by way of an interface. For example data relating to the object to be examined is taken from a database by way of the interface. A determination of the signal analysis parameter values can be implemented solely by taking the information from a database or in combination with measurements not associated with radiation exposure, for example a photographic recording, without recording a test measurement or a topogram, thereby reducing the dose exposure.

The information relating to the nature of the examination is preferably derived in an automated manner based on an examination protocol. In other words specific classification data allowing inferences to be drawn about the nature of a scheduled examination is acquired from the examination protocol and processed in an automated manner in the context of determining the signal analysis parameter values. This means in particular that no manual intervention is required when determining the signal analysis parameter values.

The automated determination of the signal analysis parameter values preferably comprises the calculation of the signal analysis parameter values based on the acquired information. A calculation here refers not only to the calculation of the signal analysis parameter values with the aid of a specific formula but also interpolation based on table values for the individual signal analysis parameters from a database or even a numerical optimization method, with a target function for example being optimized numerically.

The determination of the signal analysis parameter values can additionally be performed taking into account a parameterization of an automatic anatomical dose modulation. The dose modulation allows it to be determined whether there is much or little attenuation in the beam path in order to derive a subsequent adjustment of the tube current therefrom. However this information can also be used to change the signal analysis parameters. For example when attenuation is significant there is generally a greater degree of beam hardening, which could involve a subsequent adjustment of the energy thresholds. It is also possible to use the automatic anatomical dose modulation to infer whether or where there is an instance of high or low flux.

In an alternative variant of an embodiment of the inventive method for automatically setting signal analysis parameters of an x-ray detector the signal analysis parameters are set automatically during the imaging of the object to be examined as a function of the geometry detected during imaging and/or the current properties of the object to be examined. The setting for the signal analysis parameters can therefore be changed during the actual imaging of the object to be examined as a function of image information already acquired during imaging. This not only dispenses with the need for a prior recording for a topogram, it also allows an as it were dynamic adjustment of the signal analysis parameters based on locally different conditions in the object to be examined. It means that different values for the signal analysis parameters are determined and set for example based on local dimensions in subregions of the object to be examined and as a function of local x-ray attenuation for different subregions of the object to be examined.

An embodiment of the inventive facility for determining an adjusted setting for signal analysis parameters of an x-ray detector also advantageously has a setting unit for the automated setting of the signal analysis parameters of the x-ray detector based on the determined signal analysis parameter values. The setting unit here can for example also feature distributed components in a control facility of an x-ray system and in a detector unit. For example a setting command is generated in the control facility, the setting command being executed by an ASIC in a signal analysis unit of a detector unit, with the corresponding parameters being set in the signal analysis unit.

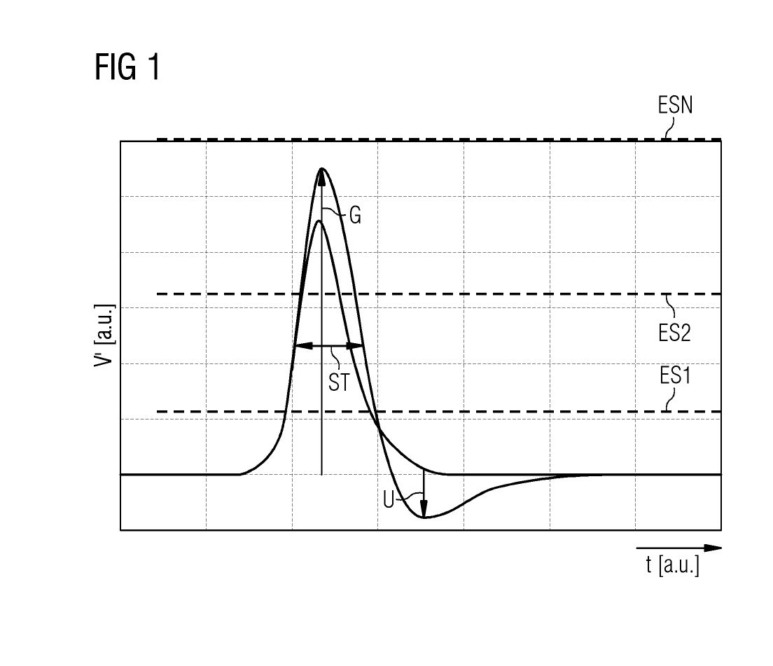

FIG. 1 shows several settable signal analysis parameters of a signal analysis unit of an x-ray detector in detail. It shows a signal generated from a charge pulse by a signal analysis unit in the form of a voltage pulse for detecting an x-ray quantum. The vertical axis shows the amplified signal voltage, in other words the output signal level, in arbitrary units (a. u.), as determined with the aid of an integration or convolution of the current pulse generated by a detection unit. The horizontal axis shows the temporal profile (in arbitrary units—a. u.) of this voltage signal. The signal analysis parameters comprise for example what is referred to as gain G, which determines the maximum signal level in relation to a normal signal. The signal analysis parameters can also comprise what is referred to as the shaping time ST, which in this instance is assumed to be the full width at half maximum of the voltage pulse. What is referred to as the undershoot U, in other words the size of the signal drop or the negative voltage according to the signal-based representation of the x-ray quantum can also be set in the form of a voltage pulse, which can be used to determine a temporal signal separation of a number of x-ray quanta. The signal analysis parameters also comprise a number N of energy thresholds, with N typically lying between two and eight, to which an energy threshold ES1, ES2, . . . , ESN is respectively assigned, for which a specific count rate value is generated in the set of count rates.

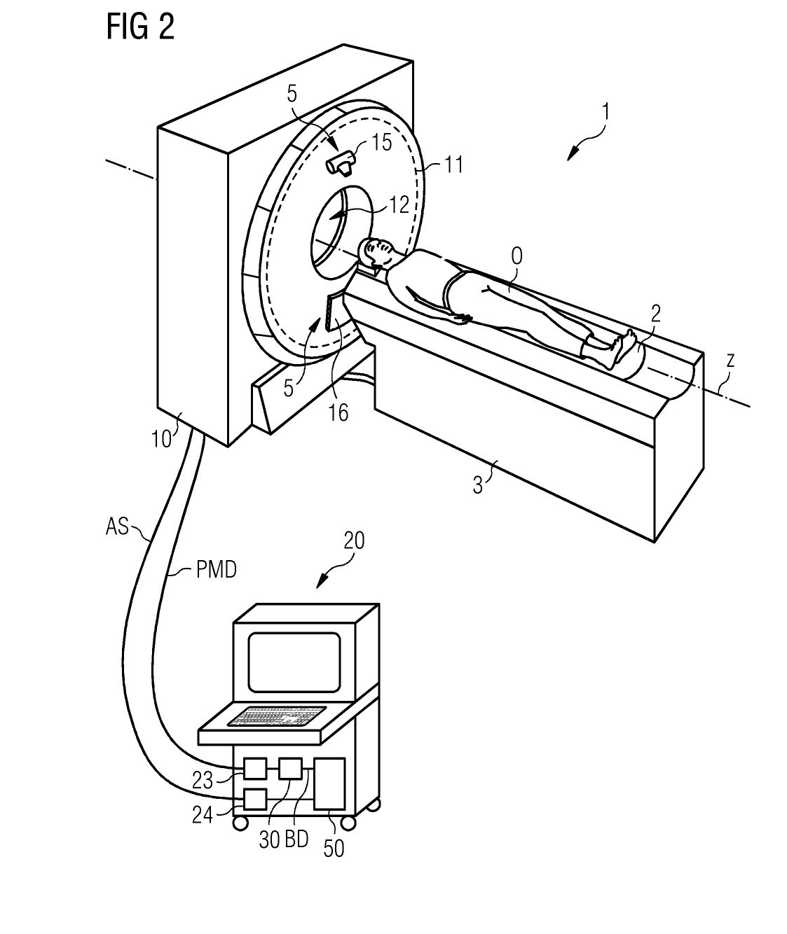

FIG. 2 shows a schematic diagram of a computed tomography system (CT system) 1 with an inventive facility 50 for the automated setting of the signal analysis parameters of an x-ray detector based on determined signal analysis parameter values according to one example embodiment of the invention.

The CT system 1 here consists essentially of a scanner 10, in which a projection data acquisition unit 5 with a detector 16 and an x-ray source 15 positioned opposite the detector 16 rotate around a measurement space 12 on a gantry 11. In front of the scanner 10 is a patient support facility 3 or a patient couch 3, the upper part 2 of which can be displaced with a patient O present thereon toward the scanner 10, in order to move the patient O through the measurement space 12 relative to the detector system 16. The scanner 10 and patient couch 3 are activated by a control facility 20, from which acquisition control signals AS are transmitted by way of a standard control interface 24, to activate the entire system according to predetermined measurement protocols in the conventional manner. The movement of the patient O along the z direction, which corresponds to the system axis z longitudinally through the measurement space 12, and the simultaneous rotation of the x-ray source 15 mean that the x-ray source 15 follows a helical path relative to the patient O during the measurement. The detector 16 always moves in a parallel manner thereto opposite the x-ray source 15, in order to acquire projection measurement data PMD which can then be used to reconstruct volume and/or layer image data. Similarly a sequential measurement method can also be performed, in which a fixed position is approached in the z direction and the required projection measurement data PMD is then acquired during a rotation, a partial rotation or a number of rotations in the relevant z position in order to reconstruct a sectional image in this z position or to reconstruct volume image data from the projection data of a number of z positions. The inventive method can also be used in principle with other CT systems, for example with a number of x-ray sources and/or detectors and/or with a detector forming a complete ring.

The projection measurement data PMD acquired by the detector 16 (hereinafter also referred to as raw data) is transferred to the control facility 20 by way of a raw data interface 23. This raw data is then processed further in an image reconstruction facility 30, which in this example embodiment is implemented in the control facility 20 in the form of software on a processor. The image reconstruction facility 30 reconstructs image data BD based on the raw data PMD with the aid of a reconstruction method. The reconstruction method used can be for example the filtered back projection method described above in the introduction.

Both the acquired raw data PMD and the image data BD as well as further information, which is input for example by way of the user interface of the control facility 20, is then forwarded to the signal analysis parameter setting facility 50.

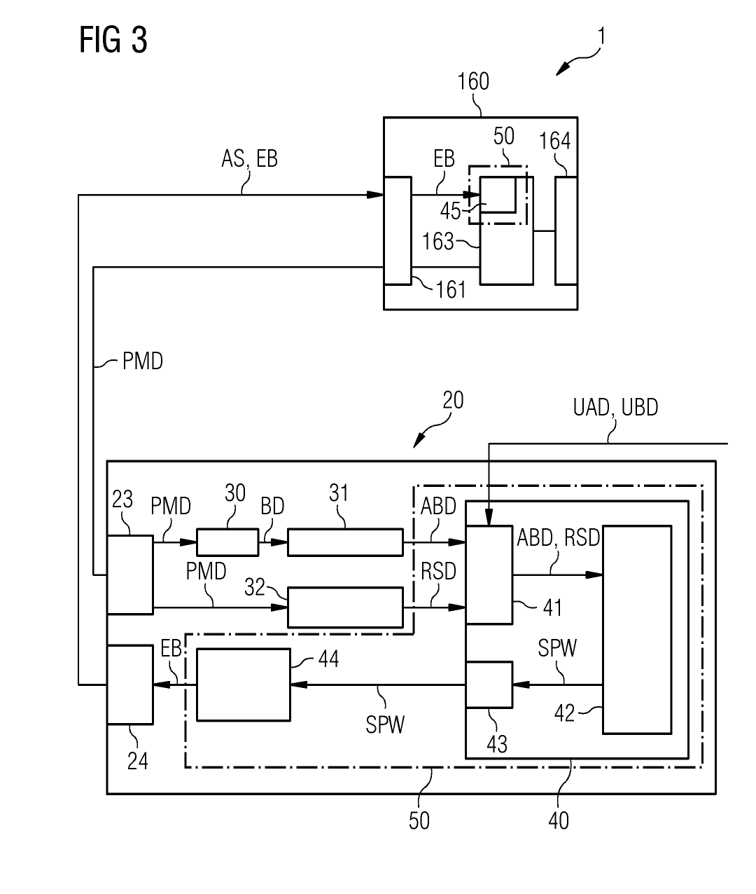

The precise structure of the control facility 20 and units interacting with it within the CT system 1 is shown in FIG. 3. This shows the control facility 20 with the signal analysis parameter setting facility 50 connected to a detection unit 160. The detection unit 160 can be for example part of the x-ray detector 16 shown in FIG. 2. An x-ray detector 16 shown in FIG. 2 can then comprise a plurality of detector units 160. The control facility 20 comprises an input interface 23, which receives projection measurement data PMD from an x-ray detector or from the detector unit 160. The projection measurement data PMD is forwarded to a reconstruction unit 30, where it is reconstructed to provide image data BD. The dimensions ABD of the patient O are determined based on the image data BD in a size determination unit 31. The x-ray attenuation RSD is also determined by an attenuation determination unit 32 based on the projection measurement data PMD. The determined data ABD, RSD is then forwarded to an input interface 41 of a signal analysis parameter determination unit 40. Additional data, for example information UAD relating to the nature of the examination and information UBD relating to the examination region of the patient O can also be transferred by way of the input interface 41 to the signal analysis parameter determination unit 40. This data can be input by a user for example or can be transferred by way of a network to the signal analysis parameter determination unit 40. The received data ABD, RSD, UAD, UBD is then forwarded to a signal analysis parameter value determination unit 42, which determines signal analysis parameter values SPW for setting the detection unit 16 based on the acquired data ABD, RSD, UAD, UBD. The determined signal analysis parameter values SPW are transferred by way of an output interface 43 of the signal analysis parameter determination facility 40 to a setting unit 44. The setting unit 44 generates a setting command EB based on the received signal analysis parameter values SPW.

The setting command EB is transferred by way of an output interface 24 of the control facility 20 to the detection unit 160. The detection unit 160 comprises an input interface 161, which receives the setting command EB from the control facility 20. The setting command EB is forwarded from the input interface 161 of the detection unit 160 to a signal analysis module 163. In the embodiment shown in FIG. 3 the input interface also functions as an output interface for acquired projection measurement data PMD. The signal analysis module 163 comprises in particular the electronic evaluation system of the detection unit 160. The signal analysis module 163 determines a set of count rates for x-ray radiation striking the detection unit 160 based on a detection signal and predetermined signal analysis parameters.

The setting of the electronic evaluation system of the signal analysis module 160 also includes the setting of the energy thresholds and other signal analysis parameters for sensor signals, for example the signal shape parameters of the signal. The setting of the electronic evaluation system is performed for example by way of a processor unit 45, which in the illustrated example embodiment is part of the signal analysis module 163. The processor unit 45, for example an

ASIC, is notionally assigned to the signal analysis parameter setting facility 50, which in this instance is therefore distributed among different separate units, in other words the x-ray detector unit 160 and the control facility 20. The x-ray detector unit 160 also features a sensor unit 164, which detects x-ray beams and triggers a sensor signal, which is forwarded to the signal analysis module 163 and evaluated there in respect of intensity or count rate and energy of the x-ray beams. As mentioned above, the setting of the signal analysis parameters, for example the energy thresholds or signal shape parameters, is performed in an automated manner based on acquired data relating to the patient and also the examination of the patient. The automated setting of the signal analysis parameters shortens the adjustment phase before the actual image recording phase, makes the adjustment precise and reduces the challenge for the operator performing the adjustment.

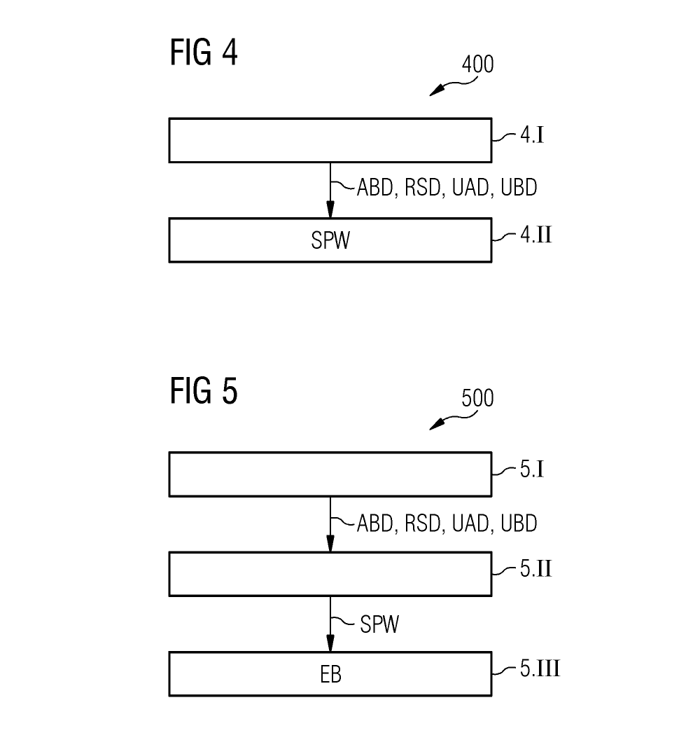

FIG. 4 shows a method 400 for the automated determination of an adjusted setting for signal analysis parameters SP of an x-ray detector 16 as a function of an object to be examined O according to one example embodiment of the invention. In step 4.I information relating to the dimensions ABD of the patient O, the x-ray attenuation RSD through the patient, the nature of the examination UAD and the examination region UBD of the patient O is first acquired. Then in step 4.II an adjusted setting for signal analysis parameters SP is determined in an automated manner based on the acquired information. More specifically the signal analysis parameter values SPW are calculated based on the acquired information. Intervention by operators during the determination of the signal analysis parameter values SPW is not required according to the invention. Setting of the x-ray detector 16 can then be performed both manually and in an automated manner.

FIG. 5 shows a method 500 for automatically setting signal analysis parameters SP of an x-ray detector 16 according to one example embodiment of the invention. In this example embodiment therefore fully automated setting of the signal analysis parameters of a detector 16 takes place. Steps 5.I and 5.II correspond to steps 4.I and 4.II. In step 5.III the signal analysis parameters SP of the x-ray detector 16 are set in an automated manner based on the determined signal analysis parameter values SPW. As shown in FIG. 3, the setting of the x-ray detector 16 can be performed with the aid of a setting facility 44 in the control facility 20, which generates a setting command EB based on the determined signal analysis parameter values SPW, the setting command EB being executed by a processor 45, which can be for example part of the electronic evaluation system 163 of the x-ray detector 16 or can be connected thereto, such that the signal analysis parameters SP of the x-ray detector 16 are set.

To conclude, it should be noted that the methods and apparatuses described above are simply preferred example embodiments of the invention and the invention can be varied by the person skilled in the art without departing from the scope of the invention, as defined by the claims. The method and setting facility have primarily been described based on a computed tomography system for recording medical image data. However the invention is limited neither to an application in computed tomography nor to an application in the medical field; in principle the invention can also be applied to other x-ray systems and also the recording of x-ray images for other purposes, for example for material testing or the like. For the sake of completeness it should also be noted that the use of the indefinite article “a” or “an” does not exclude more than one of the features in question also being present. Similarly the term “unit” or “module” does not exclude such comprising a number of components, which may also be distributed spatially.

The aforementioned description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods. Further, elements and/or features of different example embodiments may be combined with each other and/or substituted for each other within the scope of this disclosure and appended claims.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Further, at least one embodiment of the invention relates to a non-transitory computer-readable storage medium comprising electronically readable control information stored thereon, configured in such that when the storage medium is used in a controller of a magnetic resonance device, at least one embodiment of the method is carried out.

Even further, any of the aforementioned methods may be embodied in the form of a program. The program may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for automatically determining signal analysis parameter values of parameters of an x-ray detector, the method comprising:
acquiring information relating to at least one of the following groups of examination parameters
dimensions of an object to be examined,
x-ray attenuation in the object to be examined,
nature of the examination of the object to be examined, and
examination region of the object to be examined; and
automatically determining signal analysis parameter values based on the acquired information.

2. The method of claim 1, wherein the dimensions of the object to be examined includes at least one of a size and shape of the object to be examined.

3. The method of claim 1, wherein the parameters include at least one of parameters for energy thresholds and signal shape parameters.

4. The method of claim 1, wherein the acquiring of the information includes at least one of recording a topogram of the object to be examined and acquiring a photographic recording of the object to be examined.

5. The method of claim 1, wherein the acquiring of information includes weighing the object to be examined.

6. The method of claim 1, wherein the acquiring of information includes receiving data relating to the object to be examined via an interface.

7. The method of claim 1, wherein the information relating to the nature of the examination is derived in an automated manner based on an examination protocol.

8. The method of claim 1, wherein the automatically determining of the signal analysis parameter values includes calculating the signal analysis parameter values based on the acquired information.

9. The method of claim 1, wherein the automatically determining of the signal analysis parameter values is additionally performed taking into account a parameterization of an automatic anatomical dose modulation.

10. A method for automatically setting signal analysis parameters of an x-ray detector, comprising:
performing the method of claim 1; and
automatically setting the signal analysis parameters based on the determined signal analysis parameter values.

11. The method of claim 10, wherein the signal analysis parameters are set automatically during at least one of
imaging of the object to be examined as a function of the geometry detected during imaging, and
current properties of the object to be examined.

12. A facility for determining signal analysis parameter values of an x-ray detector, the facility comprising:
an input interface to acquire information relating to at least one of the following groups of examination parameters:
dimensions of an object to be examined,
x-ray attenuation in the object to be examined,
nature of the examination of the object to be examined, and
examination region of the object to be examined; and
a determination unit to automatically determine signal analysis parameter values based on the acquired information.

13. The facility of claim 12, further comprising:
a setting unit for automatically setting signal analysis parameters of the x-ray detector based on the determined signal analysis parameter values.

14. An x-ray system, comprising:
the facility of claim 12.

15. A non-transitory computer readable medium, loaded directly into a storage unit of a programmable storage facility of an x-ray system, including program segments for executing the method of claim 1 when the program is executed in the storage facility.

16. The method of claim 2, wherein the parameters include at least one of parameters for energy thresholds and signal shape parameters.

17. The method of claim 2, wherein the acquiring of the information includes at least one of recording a topogram of the object to be examined and acquiring a photographic recording of the object to be examined.

18. The method of claim 2, wherein the acquiring of information includes weighing the object to be examined.

19. The method of claim 8, wherein the automatically determining of the signal analysis parameter values is additionally performed taking into account a parameterization of an automatic anatomical dose modulation.

20. The x-ray system of claim 14, wherein the x-ray system is a computed tomography system.

21. An x-ray system, comprising:
the facility of claim 13.

22. The x-ray system of claim 21, wherein the x-ray system is a computed tomography system.

23. A non-transitory computer readable medium, loaded directly into a storage unit of a programmable storage facility of an x-ray system, including program segments for executing the method of claim 10 when the program is executed in the storage facility.
